(19) United States
(12) Patent Application Publication
(10) Pub. No.: US 2003/0083376 A1
(43) Pub. Date: May 1, 2003

(54) Depot preparations

(76) Inventors: Marcus Eh, Holzminden (DE); Horst Surburg, Holzminden (DE); Heinz-Juergen Bertram, Holzminden (DE); Steffen Sonnenberg, Holzminden (DE)

Correspondence Address: BAYER CORPORATION, PATENT DEPARTMENT, 100 BAYER ROAD, PITTSBURGH, PA, 15205, US

(21) Appl. No.: 10/222,646

(22) Filed: Aug. 16, 2002

(30) Foreign Application Priority Data
Aug. 20, 2001 (DE) 10140787.4

(51) Int. Cl. A61K 31/22; A61K 7/075

(52) U.S. Cl. 514/546

(57) ABSTRACT

The invention relates to depot preparations for the targeted release of an aldehyde or ketone together with an alcohol and a carboxylic acid, where the released aldehydes or ketones are organoleptic substances, specifically fragrances or flavorings.

### FIELD OF THE INVENTION

The invention relates to depot preparations (“delivery systems”) for the targeted release of an aldehyde or ketone, alcohol and a carboxylic acid. The released aldehydes, ketones and alcohols are organoleptic substances, specifically fragrances or flavorings. The depot preparations are obtainable by conversion of acetals into the corresponding enol ethers and subsequent reaction with a carboxylic acid, or by hydride reduction of a carboxylic ester or reaction of a carboxylic ester with an organometallic reagent and subsequent scavenging of the hemiacetal which forms with a carboxylic anhydride.

### BACKGROUND OF THE INVENTION

The method, in principle, of perfuming consumer articles involves mixing the perfume oil containing fragrances directly with the product. The problems which arise with this process are that readily volatile substances are partially or completely lost as a result of evaporation during incorporation into the product or during storage. In addition, numerous substances, and aldehydes in particular, are unstable under the given conditions, which leads to partial or complete decomposition of these molecules. The consequence of this is that all substances which underlie the problems described above can, in sensory terms, only be weakly perceived or cannot be perceived at all. In some cases this may lead to a change in the overall odor impression of the composition.

WO 94/06441 discloses acetals, ketals and specific ortho esters as depot preparations which are stable in basic media. In acidic media, such as, for example, upon contact with the skin, hydrolysis occurs, releasing alcohols or ketones.

A depot preparation for the release of aldehydes, specifically citral, in foods and here specifically in alcoholic or nonalcoholic beverages is described in WO 00/04009. These are dicarboalkoxydioxolans which are obtainable by acetylation from an aldehyde and a tartaric acid derivative. In aqueous acidic alcoholic and nonalcoholic beverages they have a very much longer half-life than comparable acetals.

WO 00/38616 claims a cyclic dioxaketone which, after hydrolysis, simultaneously releases an aldehyde or ketone and a hydroxycarboxylic acid. These compounds are prepared by reacting the corresponding hydroxycarboxylic acid with the aldehyde or ketone with the addition of catalytic amounts of acid in the water separator. Preference is given to aldehydes or ketones with fragrance properties and α-hydroxycarboxylic acids or ring-substituted benzoic acids.

However, none of the above-mentioned depot preparations has the ability to simultaneously release three components with three different functional groups. Furthermore, the problems which arise with the conventional perfuming of consumer articles (evaporation during storage, instability etc.) are solved by the depot preparation according to the present invention.

### SUMMARY OF THE INVENTION

The invention provides depot preparations comprising at least one compound of the formula (I)

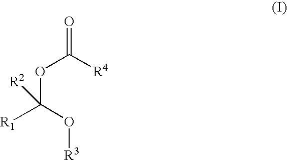

in which

R¹, R³ and R⁴, independently of one another, are an organic radical having 1 to 30 carbon atoms,

R² is hydrogen or an organic radical having 1 to 30 carbon atoms,

and the compound of the formula (I), after hydrolysis or enzymatic cleavage, releases an alcohol and a carboxylic acid in addition to the aldehyde or ketone.

### DETAILED DESCRIPTION OF THE INVENTION

In a preferred embodiment of the invention, use is made of compounds of the formula (I) in which

R¹ and R³, independently of one another, are a saturated or unsaturated, linear or branched, alicyclic or aromatic radical having 1 to 18 carbon atoms, which may optionally also contain heteroatoms,

R² is hydrogen, a saturated or unsaturated, linear or branched, alicyclic or aromatic radical having 1 to 18 carbon atoms, which may optionally also contain heteroatoms,

R⁴ is a saturated or unsaturated, linear or branched, alicyclic or aromatic radical having 1 to 22 carbon atoms, which may optionally also contain heteroatoms and which, in addition, may optionally be substituted by ionic substituents.

Preferred heteroatoms are oxygen and sulfur and more preference is given to oxygen.

Examples of ionic substituents are —CO₂M and —OCO₂M, where M is an alkali metal.

The 1-alkoxy esters of the formula (I) according to the present invention decompose on the one hand after hydrolysis in aqueous medium, preferably in acidic aqueous medium with a pH of <6, and also in alkaline aqueous medium with a pH of >9, and on the other hand after enzymatic cleavage, releasing an aldehyde or ketone, and an alcohol and a carboxylic acid which then develop their action (e.g. fragrance release) and can adhere to substrates.

Surprisingly, the release rate can be controlled via the radicals R³ and R⁴, depending on R¹ and R², such that 1-alkoxy esters with small and narrow radicals R³ and R⁴ have short half-lives, while voluminous and long-chain radicals R³ and R⁴ increase the half-life. This is significant in as much as 1-alkoxy esters of the formula (I) with different release profiles can thus, be prepared. Building on this, the depot preparation and the formulation can be matched to one another as regards respective radicals or ingredients in order to achieve an application-oriented release as a result of controlled release or controlled adhesion/transfer.

The enzymatic cleavage can preferably be carried out by esterases or lipases.

In a further preferred embodiment of the invention, the aldehyde R¹COH released from the compound of the formula (I) or the ketone R¹R²CO has a molecular weight of from 100 g/mol to 350 g/mol and more preferably from 120 g/mol to 270 g/mol. It is also preferred if the released aldehyde or the ketone is a fragrance or flavoring.

Nonlimiting examples of aldehydes which can be released following cleavage of the depot preparation according to the present invention are given below:

phenylacetaldehyde, p-methylphenylacetaldehyde, p-isopropylphenylacetaldehyde, methylnonylacetaldehyde, phenylpropanal, 3-(4-t-butylphenyl)-2-methylpropanal (lilial), 3-(4-t-butylphenyl)propanal (bourgeonal), 3-(4-methoxyphenyl)-2-methylpropanal (canthoxal), 3-(4-isopropylphenyl)-2-methylpropanal (cymal), 3-(3,4-methylenedioxyphenyl)-2-methylpropanal (helional), 3-(4-ethylphenyl)-2,2-dimethylpropanal (floralozone), phenylbutanal, 3-methyl-5-phenylpentanal, hexanal, trans-2-hexenal, cis-hex-3-enal, heptanal, cis-4-heptenal, 2-ethyl-2-heptenal, 2,6-dimethyl-5-heptenal (melonal), 2,4-heptadienal, octanal, 2-octenal, 3,7-dimethyloctanal, 3,7-dimethyl-2,6-octadien-1-al, 3,7-dimethyl-2,6-octadien-3-al, 3,7-dimethyl-6-octenal (citronellal), 3,7-dimethyl-7-hydroxyoctan-1-al (hydroxycitronellal), nonanal, 6-nonenal, 2,4-nonadienal, 2,6-nonadienal, decanal, 2-methyldecanal, 4-decenal, 9-decenal, 2,4-decadienal, undecanal, 2-methyldecanal, 2-methylundecanal, 2,6,10-trimethyl-9-undecenal (adoxal), undec-10-enylaldehyde, undec-8-enanal, dodecanal, tridecanal, tetradecanal, anisaldehyde, cinnamaldehyde, α-amylcinnamaldehyde, α-hexylcinnamaldehyde, methoxycinnamaldehyde, isocyclocitral, citronellyloxyacetaldehyde, cortexaldehyde, cuminaldehyde, cyclamenaldehyde, florhydral, heliotropin, hydratropaldehyde, vanillin, ethylvanillin, benzaldehyde, p-methylbenzaldehyde, 3,4-dimethoxybenzaldehyde, 3- and 4-(4-hydroxy-4-methylpentyl)-3-cyclohexene-1-carboxaldehyde, (lyral), 2,4-dimethyl-3-cyclohexene-1-carb-oxaldehyde (triplal), 1-methyl-3-(4-methylpentyl)-3-cyclohexene-carboxaldehyde (vernaldehyde) or p-methylphenoxyacetaldehyde (Xi aldehyde).

Nonlimiting examples of ketones which can be released following cleavage of the depot preparation according to the present invention are given below:

α-damascone, β-damascone, δ-damascone, β-damascenone, muscone, 6,7-dihydro-1,1,2,3,3-pentamethyl-4(5H)-indanone (Cashmeran), cis-jasmone, dihydrojasmone, α-ionone, β-ionone, dihydro-α-ionone, γ-methylionone, α-iso-methylionone, 4-(3,4-methylenedioxyphenyl)butan-2-one, 4-(4-hydroxyphenyl)butan-2-one, methyl βnaphthyl ketone, methyl cedryl ketone, 6-acetyl-1,1,2,4,4,7-hexamethyltetralin (tonalid), I-carvone, 5-cyclohexadecen-1-one, acetophenone, decatone, p-hydroxy-phenylbutan-2-one, 2-[2-(4-methyl-3-cyclohexenyl-1-yl)propyl]cyclopentan-2-one, 2-sec-butylcyclohexanone, β-dihydroionone, allylionone, α-irone, α-cetone, α-irisone, acetanisole, geranylacetone, 1-(2-methyl-5-isopropyl-2-cyclohexenyl)-1-propanone, acetyidiisoamylene, methylcyclocitrone, 4-t-pentylcyclohexanones, p-t-butylcyclohexanone, o-t-butylcyclohexanone, ethyl amyl ketone, ethyl pentyl ketone, menthone, methyl-7,3-dihydro-2H-1,5-benzodioxepin-3-one, fenchone.

Nonlimiting examples of alcohols which can be released following cleavage of the depot preparation according to the present invention are given below:

methanol, 2,4-dimethyl-3-cyclohexene-1-methanol (floralol), 2,4-dimethyl cyclohexanemethanol (dihydrofloralol), 5,6-dimethyl-1-methylethenylbicyclo[2.2.1]hept-5-ene-2-methanol (arbozol), 2,4,6-trimethyl-3-cyclohexene-1-methanol (isocyclogeraniol), 4-(1-methylethyl)cyclohexanemethanol (mayol), α-3,3-trimethyl-2-nor-boranemethanol, 1,1-dimethyl-1-(4-methylcyclohex-3-enyl)methanol, ethanol, 2-phenylethanol, 2-cyclohexylethanol, 2-(o-methylphenyl)ethanol, 2-(m-methylphenyl)ethanol, 2-(p-methylphenyl)ethanol, 6,6-dimethylbicyclo-[3.1.1]hept-2-ene-2-ethanol (nopol), 2-(4-methylphenoxy)ethanol, 3,3-dimethyl-Δ²-β-norbornaneethanol, 2-methyl-2-cyclohexylethanol, 1-(4-isopropylcyclohexyl)ethanol, 1-phenylethanol, 1,1-dimethyl-2-phenylethanol, 1,1-dimethyl-2-(4-methylphenyl)ethanol, n-propanol, 2-propanol, 1-phenylpropanol, 3-phenylpropanol, 2-phenylpropanol (hydrotropic alcohol), 2-(cyclododecyl)propan-1-ol (hydroxyambran), 2,2-dimethyl-3-(3-methylphenyl)propan-1-ol (majantol), 2-methyl-3-phenylpropanol, 3-phenyl-2-propen-1-ol (cinnamyl alcohol), 2-methyl-3-phenyl-2-propen-1-ol (methylcinnamyl alcohol), α-n-pentyl-3-phenyl-2-propen-1-ol (α-amylcinnamyl alcohol), ethyl 3-hydroxy-3-phenylpropionate, 2-(4-methylphenyl)-2-propanol, n-butanol, 2-butanol, 3-methylbutanol, 3-(4-methylcyclohex-3-ene)butanol, 2-methyl-4-(2,2,3-trimethyl-3-cyclopenten-1-yl)butanol, 2-ethyl-4-(2,2,3-trimethylcyclopent-3-enyl)-2-buten-1-ol, 3-methyl-2-buten-1-ol, 2-methyl-4-(2,2,3-trimethyl-3-cyclopenten-1-yl)-2-buten-1-ol, 3-hydroxy-2-butanone, ethyl 3-hydroxybutyrate, 4-phenyl-3-buten-2-ol, 2-methyl-4-phenylbutan-2-ol, 4-(4-hydroxyphenyl)butan-2-one, 4-(4-hydroxy-3-methoxyphenyl)butan-2-one, pentanol, cis-3-pentenol, 3-methylpentanol, 3-methyl-3-penten-1-ol, 2-methyl-4-phenylpentanol (pamplefleur), 3-methyl-5-phenylpentanol (phenoxanol), 2-methyl-5-phenylpentanol, 2-methyl-5-(2,3-dimethyltricyclo-[2.2.1.0(2,6)]hept-3-yl)-2-penten-1-ol (santalol), 4-methyl-1-phenyl-2-pentanol, (1-methylbicyclo[2.1.1]hepten-2-yl)-2-methylpent-1-en-3-ol, 3-methyl-1-phenylpentan-3-ol, 1,2-dimethyl-3-(1-methylethenyl)cyclopentan-1-ol, 2-isopropyl-5-methyl-2-hexenol, cis-3-hexen-1-ol, trans-2-hexen-1-ol, 2-isopropenyl-4-methyl-4-hexen-1-ol (lavandulol), 2-ethyl-2-prenyl-3-hexenol, 1-hydroxymethyl-4-iso-propenyl-1-cyclohexene (dihydrocuminyl alcohol), 1-methyl-4-isopropenylcyclohex-6-en-2-ol (carvenol), 6-methyl-3-isopropenylcyclohexan-1-ol, 1-methyl-4-isopropenylcyclohexan-3-ol, 4-isopropyl-1-methylcyclohexan-3-ol, 4-tert-butylcyclohexanol, 2-tert-butylcyclohexanol, 2-tert-butyl-4-methylcyclohexanol, 4-iso-propylcyclohexanol, 4-methyl-1-(1-methylethyl)-3-cyclohexen-1-ol, 2-(5,6,6-trimethyl-2-norbornyl)cyclohexanol, isobornylcyclohexanol, 3,3,5-trimethylcyclohexanol, 1-methyl-4-isopropylcyclohexan-3-ol, 1,2-dimethyl-3-(1-methylethyl)cyclohexan-1-ol, heptanol, 2,4-dimethylheptan-1-ol, 2,4-dimethyl-2,6-heptanedienol, 6,6-dimethyl-2-oxymethylbicyclo[3.1.1]hept-2-ene (myrtenol), 4-methyl-2,4-heptadien-1-ol, 3,4,5,6,6-pentamethyl-2-heptanol, 3,6-dimethyl-3-vinyl-5-hepten-2-ol, 6,6-dimethyl-3-hydroxy-2-methylenebicyclo[3.1.1]heptane, 1,7,7-trimethylbicyclo[2.2.1]heptan-2-ol,

2,6-dimethylheptan-2-ol, 2,6,6-trimethylbicyclo[1.3.3]heptan-2-ol, octanol, 2-octenol, 2-methyloctan-2-ol, 2-methyl-6-methylene-7-octen-2-ol (myrcenol), 7-methyloctan-1-ol, 3,7-dimethyl-6-octenol, 3,7-dimethyl-7-octenol, 3,7-dimethyl-6-octen-1-ol (citronellol), 3,7-dimethyl-2,6-octadien-1-ol (geraniol), 3,7-dimethyl-2,6-octadien-1-ol (nerol), 3,7-dimethyl-1,6-octadien-3-ol (linalool), 3,7-dimethyloctan-1-ol (pelagrol), 3,7-dimethyloctan-3-ol (tetrahydrolinalool), 2,4-octadien-1-ol, 3,7-dimethyl-6-octen-3-ol, 2,6-dimethyl-7-octen-2-ol, 2,6-dimethyl-5,7-octadien-2-ol, 4,7-dimethyl-4-vinyl-6-octen-3-ol, 3-methyloctan-3-ol, 2,6-dimethyloctan-2-ol, 2,6-dimethyloctan-3-ol, 3,6-dimethyloctan-3-ol, 2,6-dimethyl-7-octen-2-ol, 2,6-dimethyl-3,5-octadien-2-ol (muguol), 3-methyl-1-octen-3-ol, 7-hydroxy-3,7-dimethyloctanal, 3-nonanol, 2,6-nonadien-1-ol, cis-6-nonen-1-ol, 6,8-dimethylnonan-2-ol, 3-(hydroxymethyl)-2-nonanone, 2-nonen-1-ol, 2,4-nonadien-1-ol, 3,7-dimethyl-1,6-nonadien-3-ol, decanol, 9-decenol, 2-decen-1-ol, 2,4-decadien-1-ol, 4-methyl-3-decen-5-ol, 3,7,9-trimethyl-1,6-decadien-3-ol (isobutyl linalool), undecanol, 2-undecen-1-ol, 10-undecen-1-ol, 2-dodecen-1-ol, 2,4-dodecadien-1-ol, 2,7,11-trimethyl-2,6,10-dodecatrien-1-ol (farnesol), 3,7,11-trimethyl-1,6,10-dodecatrien-3-ol, 3,7,11,15-tetramethylhexadec-2-en-1-ol (phytol), 3,7,11,15-tetramethylhexadec-1-en-3-ol (isophytol), benzyl alcohol, p-methoxybenzyl alcohol (anise alcohol), para-cymen-7-ol (cuminyl alcohol), 4-methylbenzyl alcohol, 3,4-methylenedioxybenzyl alcohol, methyl salicylate, benzyl salicylate, cis-3-hexenyl salicylate, n-pentyl salicylate, 2-phenylethyl salicylate, n-hexyl salicylate, 2-methyl-5-isopropylphenol, 4-ethyl-2-methoxyphenol, 4-allyl-2-methoxyphenol (eugenol), 2-methoxy-4-(1-propenyl)phenol (isoeugenol), 4-allyl-2,6-dimethoxyphenol, 4-tert-butylphenol, 2-ethoxy-4-methylphenol, 2-methyl-4-vinylphenol, 2-isopropyl-5-methylphenol (thymol), pentyl ortho-hydroxybenzoate, ethyl 2-hydroxybenzoate, methyl 2,4-dihydroxy-3,6-dimethyl benzoate, 3-hydroxy-5-methoxy-1-methylbenzene, 2-tert-butyl-4-methyl-1-hydroxybenzene, 1-ethoxy-2-hydroxy-4-propenylbenzene, 4-hydroxytoluene, 4-hydroxy-3-methoxybenzaldehyde, 2-ethoxy-4-hydroxybenzaldehyde, decahydro-2-naphthol, 2,5,5-trimethyloctahydro-2-naphthol, 1,3,3-trimethyl-2-norbornanol (fenchol), 3a,4,5,6,7,7a-hexahydro-2,4-dimethyl-4,7-methano-1H-inden-5-ol, 3a,4,5,6,7,7a-hexahydro-3,4-dimethyl-4,7-methano-1H-inden-5-ol, 2-Methyl-2-vinyl-5-(1-hydroxy-1-methylethyl)tetrahydrofuran.

Nonlimiting examples of carboxylic acids which can be released following cleavage of the depot preparation according to the present invention are given below:

Unsubstituted saturated monocarboxylic acids (e.g. acetic acid, propionic acid, butyric acid, valeric acid, capric acid, caprylic acid, pelargonic acid, capric acid, lauric acid, myristic acid, palmitic acid, stearic acid); dialkyl-substituted acetic acids (e.g. 2-butyloctanoic acid, 2-butyldecanoic acid, 2-hexyloctanoic acid, 2-hexyldecanoic acid); mono- or polyunsaturated monocarboxylic acids (e.g. oleic acid, linoleic acid, -linolenic acid, (-linolenic acid); alkyne-, alkadiyne- or alkatriyne carboxylic acids (e.g. non-8-ynoic acid, dec-9-ynoic acid, tridec-9-ynoic acid, 13-methyltetradec-9-ynoic acid, pentadec-6-ynoic acid, pentadec-7-ynoic acid, hexadec-9-ynoic acid, 15-methylhexadec-9-ynoic acid, heptadec-2-ynoic acid, heptadec-9-ynoic acid, octadec-12-ynoic acid, octadec-6,12-diynoic acid, nonadec-9-ynoic acid); -hydroxycarboxylic acids (e.g. ∀-hydroxyvaleric acid, ∀-hydroxycaproic acid, ∀-hydroxycaprylic acid, ∀-hydroxypelargonic acid, ∀-hydroxycapric acid, ∀-hydroxylauric acid, ∀-hydroxymyristic acid, ∀-hydroxypalmitic acid, ∀-hydroxypalmitic acid); unsubstituted ∀,ω-alkanedicarboxylic acids (e.g. malonic acid, succinic acid, glutaric acid, adipic acid, pimelic acid, suberic acid, azelaic acid).

A further preferred embodiment of the depot preparation according to the present invention includes 1-alkoxy esters of the formula (I) which have poor solubility in water and thus, have a relatively large tendency to attach to substrates, or to accumulate in the headspace above an aqueous solution. In this connection, it is desirable for the 1-alkoxy esters of the formula (I) according to the present invention to have a lower solubility in water than the carboxylic acid which is released so that better attachment also of the carboxylic acid to the substrate can arise, or accumulation in the headspace above the aqueous phase takes place. Solubilities can be measured directly or can be determined more easily using octanol/water partition coefficients (log P value). The log P value is an established parameter for determining lipophilicity in the literature (A. Leo, C. Hansch and D. Elkins, Chem. Rev., 71,1971, 525-616; C. Hansch, J. E. Quinlan, G. L. Lawrence, J. Org. Chem., 33,1968, 347-350).

For molecules with a low log P value, the transition from an aqueous system to a substrate is very difficult; rather, such molecules have a tendency to dissolve and be washed away. This applies in particular to acidic compounds. The present invention can overcome this problem since the transition of the carboxylic acid with a low log P value from the aqueous solution to a substrate takes place in the chemically bonded form of the 1-alkoxy esters of the formula (I) according to the present invention having a higher log P value. This means that the transfer of a carboxylic acid following release from the depot preparation takes place at a higher rate than the transfer of the free carboxylic acid. Release takes place after subsequent cleavage of the depot preparation according to the present invention.

The controlled release of the aldehyde or ketone, of the alcohol and of the carboxylic acid from the depot preparation according to the present invention can be utilized for the treatment (e.g. fragrancing) of a wealth of substrates, such as, for example, hair, human skin, laundry and hard surfaces.

Examples of fragrances with which the depot preparation according to the present invention can be advantageously combined are given, for example, in S. Arctander, Perfume and Flavor Materials, Vol. I and II, Montclair, N.J., 1969, published privately or K. Bauer, D. Garbe and H. Surburg, Common Fragrance and Flavor Materials, 3^{rd}. Ed., Wiley-VCH, Weinheim 1997.

Individual examples which may be mentioned are:

extracts from natural raw materials such as essential oils, concretes, absolutes, resins, resinoids, balsams, tinctures, such as, for example, ambergris tincture; amyris oil; angelica seed oil; angelica root oil; aniseed oil; valerian oil; basil oil; wood moss absolute; bay oil; mugwort oil; benzoin resin; bergamot oil; beeswax absolute; birch tar oil; bitter almond oil; savoury oil; bucco leaf oil; cabreuva oil; cade oil; calmus oil; camphor oil; cananga oil; cardamom oil; cascarilla oil; cassia oil; cassia absolute; castoreum absolute; cedar leaf oil; cedarwood oil; cistus oil; citronella oil; lemon oil; copaiva balsam; copaiva balsam oil; coriander oil; costus root oil; cumin oil; cypress oil; davana oil; dill herb oil; dill seed oil; eau de brouts absolute; oakmoss absolute; elemi oil; tarragon oil; eucalyptus citriodora oil; eucalyptus oil; fennel oil; fir-needle oil; galbanum oil; galbanum resin; geranium oil; grapefruit oil; guaiac wood oil; gurjun balsam; gurjun balsam oil; helichrysum absolute; helichrysum oil; ginger oil; iris root absolute; iris root oil; jasmine absolute; calmus oil; blue camomile oil; Roman camomile oil; carrot seed oil; cascarilla oil; pine-needle oil; spearmint oil; caraway oil; labdanum oil; labdanum absolute; labdanum resin; lavandin absolute; lavandin oil; lavender absolute; lavender oil; lemongrass oil; lovage oil; distilled lime oil; pressed lime oil; linaloe oil; litsea cubeba oil; laurel leaf oil; mace oil; marjoram oil; mandarin oil; massoi bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanax oil; orange-flower absolute; orange oil; origanum oil; palmarosa oil; patchouli oil; perilla oil; Peruvian balsam oil; parsley leaf oil; parsley seed oil; petitgrain oil; peppermint oil; pepper oil; pimento oil; pine oil; pennyroyal oil; rose absolute; rosewood oil; rose oil; rosemary oil; Dalmatian sage oil; Spanish sage oil; sandalwood oil; celery seed oil; spike lavender oil; Japanese anise oil; styrax oil; tagetes oil; fir-needle oil; tea-tree oil; turpentine oil; thyme oil; Tolu balsam; tonka absolute; tuberose absolute; vanilla extract; violet leaf absolute; verbena oil; vetiver oil; juniper oil; wine lees oil; absinthe oil; wintergreen oil; ylang oil; hyssop oil; civet absolute; cinnamon leaf oil; cinnamon bark oil; and fractions thereof, or ingredients isolated therefrom;

individual fragrances from the group of hydrocarbons, such as, for example, 3-carene; α-pinene; β-pinene; α-terpinene; γ-terpinene; p-cymene; bisabolene; camphene; caryophyllene; cedrene; farnesene; limonene; longifolene; myrcene; ocimene; valencene; (E,Z)-1,3,5-undecatriene;

of aliphatic alcohols, such as, for example, hexanol; octanol; 3-octanol; 2,6-dimethylheptanol; 2-methylheptanol, 2-methyloctanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol; of aliphatic aldehydes and 1,4-dioxacycloalken-2-ones thereof, such as, for example, hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-5,9-undecadienal; heptanal diethyl acetal; 1,1-dimethoxy-2,2,5-trimethyl-4-hexene; citronellyl oxyacetaldehyde;

of aliphatic ketones and oximes thereof, such as, for example, 2-heptanone; 2-octanone; 3-octanone; 2-nonanone; 5-methyl-3-heptanone; 5-methyl-3-heptanone oxime; 2,4,4,7-tetramethyl-6-octen-3-one; of aliphatic sulfur-containing compounds, such as, for example, 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;

of aliphatic nitriles, such as, for example, 2-nonenenitrile; 2-tridecenenitrile; 2,12-tridecenenitrile; 3,7-dimethyl-2,6-octadienenitrile; 3,7-dimethyl-6-octenenitrile;

of aliphatic carboxylic acids and esters thereof, such as, for example, (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octynoate; methyl 2-nonynoate; allyl 2-isoamyloxyacetate; methyl 3,7-dimethyl-2,6-octadienoate;

of acyclic terpene alcohols, such as, for example, citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and formates, acetates, propionates, isobutyrates, butyrates, isovalerates, pentanoates, hexanoates, crotonates, tiglinates and 3-methyl-2-butenoates thereof;

of acyclic terpene aldehydes and ketones, such as, for example, geranial; neral; citronellal; 7-hydroxy-3,7-dimethyloctanal; 7-methoxy-3,7-dimethyloctanal; 2,6,10-trimethyl-9-undecenal; geranylacetone; and the dimethyl and diethyl acetals of geranial, neral, 7-hydroxy-3,7-dimethyloctanal;

of cyclic terpene alcohols, such as, for example, menthol; isopulegol; alpha-terpineol; terpinenol-4; menthan-8-ol; menthan-1-ol; menthan-7-ol; borneol; isoborneol; linalool oxide; nopol; cedrol; ambrinol; vetiverol; guaiol; and formates, acetates, propionates, isobutyrates, butyrates, isovalerates, pentanoates, hexanoates, crotonates, tiglinates and 3-methyl-2-butenoates thereof;

of cyclic terpene aldehydes and ketones, such as, for example, menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalen-8(5H)-one; nootkatone; dihydronootkatone; alpha-sinensal; beta-sinensal; acetylated cedarwood oil (methyl cedryl ketone);

of cyclic alcohols, such as, for example, 4-tert-butylcyclohexanol; 3,3,5-trimethylcyclohexanol; 3-isocamphylcyclohexanol; 2,6,9-trimethyl-Z2,Z5, E9-cyclododecatrien-1-ol; 2-isobutyl-4-methyltetrahydro-2H-pyran-4-ol;

of cycloaliphatic alcohols, such as, for example, alpha-3,3-trimethylcyclo-hexylmethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;

of cyclic and cycloaliphatic ethers, such as, for example, cineol; cedryl methyl ether; cyclododecyl methyl ether; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methyl propyl)-1,3-dioxane;

of cyclic ketones, such as, for example, 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4(5H)-indanone; 5-cyclohexadecen-1-one; 8-cyclohexadecen-1-one; 9-cycloheptadecen-1-one; cyclopentadecanone;

of cycloaliphatic aldehydes, such as, for example, 2,4-dimethyl-3-cyclohexenecarbaldehyde; 2-methyl-4-(2,2,6-trimethyl-cyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;

of cycloaliphatic ketones, such as, for example, i-(3,3-dimethylcyclohexyl)-4-penten-1-one; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl-2,6,10-trimethyl-2,5,9-cyclododecatrienyl ketone; tert-butyl 2,4-dimethyl-3-cyclohexen-1-yl ketone;

of esters of cyclic alcohols, such as, for example, 2-tert-butylcyclohexyl acetate; 4-tert-butylcyclohexyl acetate; 2-tert-pentylcyclohexyl acetate; 4-tert-pentylcyclohexyl acetate; decahydro-2-naphthyl acetate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5 or 6-indenyl acetate; 4,7-methano-3a,4,5,6,7,7a-hexahydro-5 or 6-indenyl propionate; 4,7-methano-3a,4,5,6,7,7a-hexahydro-5 or 6-indenyl isobutyrate; 4,7-methanooctahydro-5 or 6-indenyl acetate;

of esters of cycloaliphatic carboxylic acids, such as, for example, allyl 3-cyclohexyl-propionate; allyl cyclohexyloxyacetate; methyl dihydrojasmonate; methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolan-2-acetate;

of aromatic hydrocarbons, such as, for example, styrene and diphenylmethane;

of araliphatic alcohols, such as, for example, benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;

of esters of araliphatic alcohols and aliphatic carboxylic acids, such as, for example, benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate; of araliphatic ethers, such as, for example, 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;

of aromatic and araliphatic aldehydes, such as, for example, benzaldehyde; phenylacetaldehyde; 3-phenylpropanal; hydratropaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;

of aromatic and araliphatic ketones, such as, for example, acetophenone; 4-methylacetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′, 5′, 6′, 8′,8′-hexamethyl-2-aceto-naphthone;

of aromatic and araliphatic carboxylic acids and esters thereof, such as, for example, benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;

of nitrogen-containing aromatic compounds, such as, for example, 2,4,6-trinitro-1,3-dimethyl-5-tert-butylbenzene; 3,5-dinitro-2,6-dimethyl-4-tert-butylacetophenone; cinnamonitrile; 5-phenyl-3-methyl-2-pentenenitrile; 5-phenyl-3-methylpentanenitrile; methyl anthranilate; methyl N-methylanthranilate; Schiff bases of methyl anthranilate with 7-hydroxy-3,7-dimethyloctanal; 2-methyl-3-(4-tert-butylphenyl)propanal or 2,4-dimethyl-3-cyclohexenecarbaldehyde; 6-isopropylquinoline; 6-isobutylquinoline; 6-sec-butylquinoline; indole; skatole; 2-methoxy-3-isopropylpyrazine; 2-isobutyl-3-methoxypyrazine;

of phenols, phenyl ethers and phenyl esters, such as, for example, estragole; anethole; eugenole; eugenyl methyl ether; isoeugenole; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenylacetate;

of heterocyclic compounds, such as, for example, 2,5-dimethyl-4-hydroxy-2H-fu ran-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;

of lactones, such as, for example, 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

Fragrances or perfume oils which comprise the depot preparation according to the present invention can be used in liquid form, undiluted or diluted with a solvent for perfumings. Suitable solvents here are, for example, ethanol, isopropanol, diethylene glycol monoethyl ether, glycerol, propylene glycol, 1,2-butylene glycol, dipropylene glycol, diethyl phthalate, triethyl citrate, isopropyl myristate etc.

In addition, fragrances or perfume oils which contain the depot preparation according to the present invention can be adsorbed to a carrier substance which serves both to distribute fragrances finely within the product and to release them in a controlled manner during use. Such carriers may be porous in organic materials, such as light sulfate, silica gels, zeolites, gypsums, clays, clay granules, gas concrete etc., or organic materials such as wood and cellulose-based substances.

Fragrances or perfume oils which comprise the depot preparation according to the present invention can also be microencapsulated, spray-dried, in the form of inclusion complexes or in the form of extrusion products and can be added in this form to the product to be perfumed.

The properties of the fragrances or perfume oils modified in this way can optionally be further optimized by “coating” with suitable materials with regard to a more targeted scent release, for which purpose preference is given to using wax-like synthetic materials, such as, for example, polyvinyl alcohol.

The microencapsulation of the fragrances or perfume oils can, for example, be carried out by the “coacervation process” using capsule materials made from, for example, polyurethane-like substances or soft gelatines. The spray-dried perfume oils can, for example, be prepared by spray drying an emulsion or dispersion comprising the perfume oil, where the carriers used can be modified starches, proteins, dextrin and vegetable gums. Inclusion complexes can be prepared, for example, by introducing dispersions of the perfume oil and cyclodextrins or urea derivatives into a suitable solvent, e.g. water. Extrusion products can be obtained by melting the perfume oils with a suitable wax-like substance and by extrusion with subsequent solidification, optionally in a suitable solvent, e.g. isopropanol.

In perfume compositions, the amount of depot preparation according to the present invention used is 0.01 to 75% by weight, preferably 0.05 to 50% by weight, more preference being given to a use amount of from 0.5 to 20% by weight, based on the overall perfume oil.

Fragrances or perfume oils which comprise the depot preparation according to the invention can be used in concentrated form, in solutions or in above-described modified form for the preparation of, for example, cosmetic care products. In this case, and particularly for washing products or other product applications, a long-lasting odor impression on the skin or the hair is desired. Examples which may be mentioned here are: perfume extraits, eaux de parfums, eaux de toilettes, aftershaves, eaux de Colognes, preshave products, splash colognes, body care compositions such as, for example, solid and liquid soaps, shower gels, shampoos, shaving soaps, shaving foams, bath oils, cosmetic emulsions of the oil-in-water type, the water-in-oil type and the water-in-oil-in-water type, such as, for example, skin creams and lotions, face creams and lotions, sunscreen creams and lotions, aftersun creams and lotions, hand creams and lotions, foot creams and lotions, depilatory creams and lotions, aftershave creams and lotions, tanning creams and lotions, hair care products such as, for example, hairsprays, hair gels, setting hair lotions, hair rinses, permanent and semipermanent hair colorants, hair shaping compositions, such as cold waves and hair-smoothing compositions, hair tonics, hair creams and lotions, deodorants and antiperspirants, such as, for example, underarm sprays, roll-ons, deodorant sticks, deodorant creams, products for decorative cosmetics, such as, for example, eyeshadows, nail varnishes, make-up, lipsticks, mascara.

In addition, fragrances or perfume oils which comprise the depot preparation according to the present invention can be used in concentrated form, in solutions or in the above-described modified form for the preparation of, for example, household products, such as floor cleaners, window cleaners, dishwashing detergents, bath and sanitary cleaners, scouring milk, solid and liquid WC cleaners, pulverulent and foam carpet cleaners, liquid laundry detergents, pulverulent laundry detergents, laundry pretreatment agents such as bleaches, soaking agents and stain removers, fabric softeners, washing soaps, washing tablets, disinfectants, surface disinfectants.

Moreover, the depot preparation according to the present invention can be used for the aromatization of, for example, packing products or foods, and use forms thereof for the use as food for human or animal consumption.

In particular, preferred products to be aromatized are, for example, confectionery, bakery goods, chocolates, gelatine goods, sweets, alcoholic beverages, nonalcoholic beverages, ice cream, yoghurt, milk drinks, soups, sauces, snacks, chewing gum, mouthwash, meat and sausage goods, vegetable protein goods, fish, cheese and baby food.

The 1-alkoxy esters of the formula (I) according to the present invention can be prepared by the methods well known to the person skilled in the art. In a first synthesis route, the aldehyde R¹CHO or the ketone R¹R²CO is reacted with 2 to 4 equivalents of alcohol R³OH in the presence of catalytic amounts of acid to give the corresponding acetal (J. A. Barth, Organikum, 20th Edition, J. A. Barth Verlag, Leipzig 1996). Then, under acidic catalysis, one equivalent of alcohol is cleaved off from the acetal, thus giving the corresponding enol ether. Acids which can be used for the alcohol cleavage to give the enol ethers are, for example, trimethylsilyl, trimethylsulfonate (Gassman, P. G.; Burns, S. J.; Pfister, K. B., J. Org. Chem., 58, 1449-1457,1993), boron trifluoride (Faja, M.; Reese, C. B.; Song, Q.; Zhang, P.-Z., J. Chem. Soc. Perkin Trans. 1,191-194, 1997), p-toluenesulfonic acid (Greco, C. V.; Grosso, V. G., J. Org. Chem., 38, 146-151, 1973), sulfuric acid (Eliel, E. L; Krishnamurthy, S., J. Org. Chem., 30, 848-854,1965), iron chloride (Fadel, A.; Yefsah, R.; Salaun, J., Synthesis, 37-39,1987) and phosphoric acid (Ferwanah, A.; Pressler, W.; Reichardt, C., Tetrahedron Lett., 40, 3979-3982, 1973). Subsequent acid-catalysed reaction of the enol ether with a carboxylic acid R⁴CO₂H produces the 1-alkoxy esters (Boons, G.-J.; Downham, R.; Kim, K. S.; Ley, S. V.; Woods, M., Tetrahedron, 50, 7157-7176,1994).

In a second synthesis route for the preparation of the 1-alkoxy esters of the formula (I) according to the present invention, a carboxylic ester R¹CO₂R³ is reacted with an aluminum hydride reducing agent, preferably diisobutylaluminum hydride, at a temperature T=<0° C., preferably T=<−60° C., and then a carboxylic anhydride (R⁴CO)₂O is added in order to scavenge the hemiacetal which forms (Kopecky, D. J.; Rychnovsky, S. D., J. Org. Chem., 65, 191-198, 2000). In addition, the carboxylic ester R¹CO₂R³ can be reacted with an organometallic reagent R²M at a temperature T=<0° C., preferably T=<−60° C., and then the hemiacetal which forms is scavenged with a carboxylic anhydride (R⁴CO)₂O (Hayakawa, H.; Miyazawa, M.; Tanaka, H.; Miyasaka, T., Nucleosides Nucleotides, 13, 297-308, 1994).

### EXAMPLES

The following nonlimiting examples illustrate the invention.

1. General procedure for the preparation of the 1-alkoxy esters 0.65 mol of alcohol and 0.16 mol of aldehyde are introduced into 150 ml of toluene, and 16 mmol of citric acid are added thereto. The mixture is then heated on a water separator until visible amounts of water no longer separate off. When the reaction is complete, the toluene and the excess alcohol are distilled off, the residue is taken up in ether, and the organic phase is washed twice with saturated NaHCO₃ solution. The resulting acetal can be used without further purification in the subsequent reaction.

100 mmol of acetal and 180 mmol of N,N′-diisopropylamine are introduced into 150 ml of CH₂Cl₂ and cooled to −20° C. Then, 165 mmol of trimethylsilyl trifluoromethanesulfonate are added thereto such that the temperature does not exceed −15° C. When the dropwise addition is complete, the mixture is allowed to reach room temperature and is stirred until the reaction is complete. 200 ml of 1 M NaOH are then added, the mixture is stirred for 5 minutes and the phases are separated. The organic phase is washed twice more with 1 M NaOH, then dried, filtered and concentrated by evaporation. The crude enol ether is purified by means of chromatography over silica gel.

20 mmol of the enol ether and 25 mmol of the carboxylic acid are introduced into 30 ml of toluene and cooled to 0° C. 0.02 mmol of p-toluenesulfonic acid is then added, the mixture is stirred for 2 h at the above temperature and then cooling is removed. When the reaction is complete, the reaction solution is diluted with diethyl ether and washed twice with saturated NaHCO₃ solution. The organic phase is then dried, filtered and concentrated by evaporation. The crude 1-alkoxy ester is purified by means of chromatography over silica gel.

2. General procedure for the preparation of the 1-alkoxy esters 10 mmol of carboxylic ester are introduced into 60 ml of dichloromethane at −78° C., and 20 mmol of diisobutylaluminum hydride are added dropwise such that the temperature does not exceed −65° C. 30 mmol of pyridine are then added, followed by 20 mmol of N,N-dimethylaminopyridine, dissolved in 30 ml of dichloromethane, such that the temperature does not exceed −65° C. 60 mmol of carboxylic anhydride are then added and the mixture is stirred for a further 16 hours at −78° C. The reaction solution is then allowed to warm to 0° C. and then stirred for a further 30 minutes at 0° C., and then 100 ml of cold, saturated NH₄Cl solution are added at 0° C. The mixture is then allowed to reach room temperature, the phases are separated and the aqueous phase is extracted with 3×150 ml of dichloromethane. The combined organic phases are washed again with saturated NaHCO₃ solution and saturated NaCl solution, then dried with Na₂SO₄, filtered and freed from solvent on a rotary evaporator. The crude 1-alkoxy ester is purified by means of chromatography over silica gel.

### Example 1

3-(4-tert-Butylphenyl)-1-methoxypropyl Nonanoate

¹H-NMR (200 MHz, CDCl₃): δ (ppm)=0.87 (t, J=6.5 Hz, 3H), 1.20-1.38 (m, 10H), 1.31 (s, 9H), 1.52-1.70 (m, 2H), 1.94-2.07 (m, 2H), 2.28-2.37 (m, 2H), 2.61-2.72 (m, 2H), 3.42 (s, 3H), 5.78 (t, J=5.4 Hz, 1H), 7.12 (d, J=8.3 Hz, 2H), 7.31 (d, J=8.3 Hz, 2H).

¹³C-NMR (50 MHz, CDCl₃): δ (ppm)=14.1, 22.6, 24.9, 29.1, 29.2, 29.3, 29.5, 31.3 (3C), 31.7, 34.3, 34.4, 35.7, 56.5, 98.9, 125.2 (2C), 127.8 (2C), 137.8, 148.6, 173.6.

### Example 2

3-(4-tert-Butylphenyl)-1-methoxypropyl Heptanoate

¹H-NMR (200 MHz, CDCl₃): δ (ppm)=0.88 (t, J=6.8 Hz, 3H), 1.21-1.38 (m, 6H), 1.31 (s, 9H), 1.55-1.72 (m, 2H), 1.94-2.07 (m, 2H), 2.28-2.37 (m, 2H), 2.61-2.72 (m, 2H), 3.42 (s, 3H), 5.78 (t, J=5.3 Hz, 1H), 7.11 (d, J=8.6 Hz, 2H), 7.33 (d, J=8.6 Hz, 2H).

¹³C-NMR (50 MHz, CDCl₃): δ (ppm)=14.0, 22.5, 24.9, 28.8, 29.5, 31.3 (3C), 31.4, 34.3, 34.4, 35.8, 56.5, 98.9, 125.2 (2C), 127.8 (2C), 137.8, 148.6, 173.5.

### Example 3

1-[(2-Ethyl hexyl)oxy]ethyl Nonanoate

¹H-NMR (200 MHz, CDCl₃): δ (ppm)=0.82-0.94 (m, 9H), 1.22-1.40 (m, 18H), 1.38 (d, J=5.2 Hz, 3H), 1.55-1.72 (m, 3H), 2.32 (t, J=7.5 Hz, 2H), 3.42 (ddd, J=24.2,5.7, 1.9 Hz, 1H), 3.46 (ddd, J=24.2, 5.5, 1.7 Hz, 1H), 5.90 (q, J=5.2 Hz, 1H).

¹³C-NMR (50 MHz, CDCl₃): δ (ppm)=10.9,14.1 (2C), 20.7, 22.6, 23.0, 23.6, 24.9, 29.0, 29.1 (2C), 29.2, 30.3, 31.8, 34.6, 39.4, 71.7, 96.4,173.5.

### Example 4

1-[(2-Ethyl hexyl)oxy]ethyl Heptanoate

¹H-NMR (200 MHz, CDCl₃): δ (ppm)=0.81-0.94 (m, 9H), 1.22-1.39 (m, 14H), 1.39 (d, J=5.3 Hz, 3H), 1.55-1.72 (m, 3H), 2.31 (t, J=7.4 Hz, 2H), 3.33 (ddd, J=28.5, 9.2, 3.2 Hz, 1H), 3.46 (ddd, J=28.5, 9.2, 3.5 Hz, 1H), 5.90 (q, J=5.2 Hz, 1H).

¹³C-NMR (50 MHz, CDCl₃): δ (ppm)=11.0, 14.0, 14.1, 20.7, 22.5, 23.0, 23.6, 24.9, 28.8, 29.0, 30.3, 31.5, 34.6, 39.4, 71.7, 96.3, 173.4.

### Example 5

1-(2-Phenylethoxy)heptyl Acetate

¹H-NMR (200 MHz, CDCl₃): δ (ppm)=0.87 (t, J=6.5 Hz, 3H), 1.22-1.34 (m, 8H), 1.58-1.72 (m, 2H), 2.05 (s, 3H), 2.90 (t, J=7.2 Hz, 2H), 3.68 (ddd, J=9.6, 7.3, 6.7 Hz, 1H), 3.88 (ddd, J=9.6, 7.7, 7.1 Hz, 1H), 5.81 (t, J=5.5 Hz, 1H), 7.18-7.30 (m, 5H).

¹³C-NMR (50 MHz, CDCl₃): δ (ppm)=14.0, 21.2, 22.5, 23.8, 28.8, 31.6, 34.4, 36.0, 70.1, 98.6, 126.1, 128.2 (2C), 128.8 (2C), 138.2, 170.8.

### Example 6

1-(2-Phenylethoxy)heptyl Propionate

¹H-NMR (200 MHz, CDCl₃): δ (ppm)=0.87 (t, J=6.5 Hz, 3H), 1.13 (t, J=7.6 Hz, 3H), 1.20-1.34 (m, 8H), 1.58-1.72 (m, 2H), 2.33 (q, J=7.6 Hz, 2H), 2.88 (t, J=7.2 Hz, 2H), 3.68 (ddd, J=7.1, 7.5, 9.5 Hz, 1H), 3.87 (ddd, J=6.6, 7.4, 9.5 Hz, 1H), 5.83 (t, J=5.5 Hz, 1H), 7.15-7.34 (m, 5H).

¹³C-NMR (50 MHz, CDCl₃): δ (ppm)=9.0, 14.0, 22.5, 23.9, 27.7, 28.9, 31.6, 34.4, 36.1, 70.1, 98.5,126.1,128.2 (2C), 128.8 (2C), 138.3, 174.1.

The depot preparations according to the present invention were incorporated into numerous consumer products and their performance properties were investigated using various methods. For the preparation of the formulations for the consumer products, molar equivalents of the aldehydes/ketones and alcohols were used in the form of the 1-alkoxy esters or in the form of the free aldehydes/ketones and alcohols in order to ensure comparability.

Method 1: Storage Stability

The storage stability of a fragrance or of a depot preparation is defined by the percentage of substance still present after storage.

$$\frac{\text{Amount after storage}}{\text{Amount before storage}} * 100 \% = \text{Storage stability [\%]}$$

For the determination and for the comparison of the storage stability the depot preparation (DP) containing one or more 1-alkoxy esters and the corresponding aldehydes/ketones and alcohols (AL) are incorporated into separate samples (S_{DP} and S_{AL}) of the same formulation of a consumer product, such as, for example, laundry detergent, shampoo or soap. The separate samples are then divided into portions. One portion of the sample S_{DP} and S_{AL} is subjected immediately to a suitable extraction and analytical measurement in order to determine the amount of depot preparation or aldehyde/ketone and alcohol prior to storage. In the analytical investigation by, for example, gas chromatography, a suitable standard is used for quantification. The second portion is subjected to storage at elevated temperature for a defined period and then extracted and quantified using the same methods.

Method 2: Odor Evaluation

The odor evaluation of a depot preparation per se or in comparison with the corresponding aldehydes/ketones and alcohols is carried out by a group of trained individuals. Here, the odor strength of the consumer product in use with regard to the aldehyde/ketone and alcohol used is assessed. The consumer product is used according to its designation for application to the skin or for the washing of laundry, skin or hair. During use, the consumer product itself, its aqueous solutions, the damp or dry laundry or the damp or dry skin, for example, is then assessed in terms of odor on a scale from 1 (weak odor) to 6 (strong odor).

Method 3: Headspace Release Rate

The analytical measurement of the concentration of fragrances themselves, the depot preparations and the fragrances released from the depot preparations is carried out by means of gas chromatography. In this connection, various injection methods, such as, for example, thermodesorption, liquid injection and gas injection, can also be used.

Prior to the analytical measurement of fragrances, various enrichment methods, such as, for example, extraction, concentration or adsorption, can be used. Suitable extractants for liquid-liquid or liquid-solid extractions are, for example, solvents such as, for example, carbon dioxide, ethers, ketones, hydrocarbons, alcohols, water and esters.

In addition, by freezing a static or dynamic headspace above the perfumed product or substrates treated with the perfumed product, such as hair, textiles or skin, by means of cool traps, an enrichment or concentration can be achieved.

For the adsorption or extraction of fragrances from a static or dynamic headspace, surface-active adsorbents such as, for example, hair, textiles, ceramic, plastic, poly-2,6-diphenyl-p-phenylene oxide (Tenax®), crosslinked porous polymers based on styrene, ethylvinylbenzene, vinylpyrrolidone, vinylpyrridine and ethylene glycol dimethacrylate (Poropax® series) and activated carbon, are suitable. The fragrances enriched on these adsorbents are then desorbed by heating (thermodesorption) or using solvents and can then be analyzed.

### Example 7

### Powder Laundry Detergents

The laundry detergents A and B were used for washing or examined analytically both directly and after storage for four weeks.

TABLE 1
Powder laundry detergent formulation

| Ingredients | | A | B |
|---|---|---|---|
| Powder laundry | | 99.7 | 99.78 |
| detergent | | | |
| DP Example 5 (4) | 1-(2-Phenylethoxy)heptyl acetate | 0.3 | |
| Aldehyde C7 (4) | n-Heptanal | | 0.12 |
| Phenylethyl alcohol | 2-Phenylethanol | | 0.13 |
| (4) | | | |

Odor Assessment

Cotton fabric and synthetic fabric were washed together in two different machines at the same time with the above-mentioned laundry detergents A and B and evaluated in terms of odor in the damp and in the dry state: the odor strength of the items of laundry which were washed with the laundry detergent A containing depot preparation was significantly higher than the odor strength of the items of laundry which were washed with the laundry detergent B containing the free aldehyde and free alcohol.

Storage Stability

The storage stability of the free aldehyde after one month was 27%. The acid formed from the free aldehyde produced an unpleasant odor note. The storage stability of the free alcohol was 91%. The storage stability of the aldehyde and of the alcohol in the depot preparation was 93% and was thus significantly higher.

Release Rate

The washed textiles A and B were each transferred to separate glass bottles. SPME analysis (“Solid Phase Microextraction”) or direct headspace analysis was then used to analyse the relative concentration of free aldehyde and free alcohol. More than five times more free aldehyde and free alcohol was found in the headspace above the damp laundry washed with the laundry detergent A. Seven times more aldehyde and alcohol was found in the headspace above the dry laundry washed with the laundry detergent A.

### Example 8

### Soap

The soap formulation below can be prepared in accordance with generally known methods. The data refer to percentages by weight. The resulting soaps A and B were used for washing or examined analytically both directly and also after storage for four weeks.

TABLE 2
Soap formulation

| Ingredients | | A | B |
|---|---|---|---|
| Soap base (1) | Sodium Tallowate | 75.0 | 75.0 |
| Soap base (1) | Sodium Cocoate | 10.0 | 10.0 |
| Water | Water | 13.0 | 13.0 |
| Bayertitan AZ (2) | Titanium Dioxide | 0.3 | 0.3 |
| Tinopal CBS-X (3) | Disodium Distyrylbiphenyl | 0.2 | 0.2 |
| | Disulfonate | | |
| DP Example 5 (4) | 1-(2-Phenylethoxy)heptyl acetate | 0.3 | |
| Aldehyde C7 (4) | n-Heptanal | | 0.12 |
| Phenylethyl alcohol (4) | 2-Phenylethanol | | 0.13 |

Suppliers
(1) Enzian Seifenfabrik, 72555 Metzingen, Germany
(2) Bayer AG, Bayerwerk, D-51368 Leverkusen, Germany
(3) Ciba Spezialitätenchemie AG, 4000 Basle, Switzerland
(4) Haarmann & Reimer GmbH, D-37603 Holzminden, Germany
(5) Quest International, Ashford, England

Odor and Color Assessment

The soap formulations A and B were stored for about three months at room temperature.

Soap A which comprises the depot preparation showed no or only a slight color change while soap B had a yellowish or grey coloration, respectively. Color stability is achieved as a result of the use of the depot preparation.

After storage, 1 g of each of the soaps was dissolved in 100 g of hand-hot water, and the bars of soaps were used for washing skin.

In all cases the scent impression above the aqueous solutions of soap A which comprises the depot preparation was significantly stronger than the scent impression of soap B which comprises the free aldehyde and alcohol.

The scent impression of the washed skin which was washed with soap A was likewise greater and longer lasting than the scent impression after washing with soap B.

Storage Stability

The soap formulations A and B were stored in the dark for about one month at room temperature. The depot preparation exhibited a significantly greater storage stability than the corresponding aldehyde.

TABLE 3
Storage stability of depot preparation and free aldehyde in soap

| Ingredients | | Storage stability [%] |
|---|---|---|
| DP Example 5 (4) | 1-(2-Phenylethoxy)heptyl | 93 |
| | acetate | |
| Aldehyde C7 (4) | n-Heptanal | 27 |
| Phenylethyl alcohol | 2-Phenylethanol | 91 |
| (4) | | |

Release Rate

To determine the hydrolysis rate, the depot preparation was added to a 1% strength aqueous soap solution, and the concentration of the depot preparation or of the free aldehyde was measured by SPME headspace analysis.

The depot preparation in soap A exhibited spontaneous hydrolysis relative to the corresponding aldehyde and alcohol. After just 5 minutes the depot preparation had completely hydrolyzed.

Through the suitable choice of the radicals in the depot preparation, a delayed or incomplete hydrolysis can also be achieved. As a result, some of the depot preparation can then be applied to the skin in the washing process, and then, as a result of the slower cleavage of the 1-alkoxy esters, a long-lasting scent impression on the skin can be achieved.

### Example 9

### Shampoo

The following shampoo formulation can be prepared by generally known methods. The data refer to percentages by weight.

TABLE 4
Shampoo formulation

| Ingredients | | A | B |
|---|---|---|---|
| Plantacare PS 10 (1) | Sodium Laureth Sulphate | 20.000 | 20.000 |
| | (and) Lauryl Glycoside | | |
| Demineralized water | Water (Aqua) | ad 100% | ad 100% |
| Sodium chloride | Sodium Chloride | 1.400 | 1.400 |
| Citric acid 10.0% | Citric Acid | 1.650 | 1.650 |
| solution | | | |
| Phenonip (2) | Phenoxyethanol (and) | 0.500 | 0.500 |
| | Methylparaben (and) | | |
| | Ethylparaben (and) | | |
| | Propylparaben (and) | | |
| | Butylparaben | | |
| DP Example 5 (4) | 1-(2-Phenylethoxy)heptyl | 0.3 | |
| | acetate | | |
| Aldehyde C7 (4) | n-Heptanal | | 0.12 |
| Phenylethyl alcohol | 2-Phenylethanol | | 0.13 |
| (4) | | | |

Odor Assessment

The resulting shampoos A and B were used for washing hair tresses or in a half-head washing test on test persons. In the half-head washing test, one half of the hair of the test persons was in each case washed with shampoo A, and the other half of the hair was washed with shampoo B.

The hair tresses washed with shampoo B had a slightly higher odor strength after washing compared with the hair tresses which were washed with shampoo A. However, the odor intensity of the hair washed with shampoo B decreased rapidly and was no longer distinguishable from a neutral sample after about four hours.

The same scent evaluation was also obtained for the half-head washing test on test persons. However, a long-lasting aldehyde and alcohol odor was perceived from the half which was washed with shampoo A. Even after a few days a significant aldehyde and alcohol odor was perceivable on the hair half A. As a result of the greater transfer and subsequent slow cleavage of the depot preparation relative to the free aldehyde, a long-lasting scent impression can be achieved. This demonstrates the advantage of the depot preparation compared with the free aldehyde and free alcohol in the application.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Depot preparations comprising at least one compound of the formula (I) 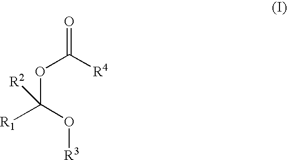
in which
R¹, R³ and R⁴, independently of one another, are an organic radical having 1 to 30 carbon atoms,
R² is hydrogen or an organic radical having 1 to 30 carbon atoms,
and the compound of the formula (I), after hydrolysis or enzymatic cleavage, releases an alcohol and a carboxylic acid in addition to the aldehyde or ketone.

2. Depot preparations according to claim 1, wherein, in formula (I)
R¹ and R³, independently of one another, are a saturated or unsaturated, linear or branched, alicyclic or aromatic radical having 1 to 18 carbon atoms,
R² is hydrogen, a saturated or unsaturated, linear or branched, alicyclic or aromatic radical having 1 to 18 carbon atoms,
R⁴ is a saturated or unsaturated, linear or branched, alicyclic or aromatic radical having 1 to 22 carbon atoms.

3. Depot preparations according to claim 1, wherein the radicals R¹ and R² release an aldehyde R¹COH or a ketone R¹R²CO which have a molecular weight of from 100 g/mol to 350 g/mol.

4. Depot preparations according to claim 3, wherein the radicals R¹ and R² release an aldehyde R¹COH or a ketone R¹R²CO which have a molecular weight of from 120 g/mol to 270 g/mol.

5. Fragrances or perfume oils comprising
a) depot preparations comprising at least one compound of the formula (I) 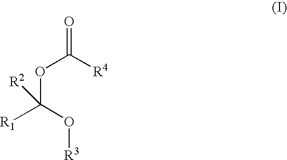
in which
R¹, R³ and R⁴, independently of one another, are an organic radical having 1 to 30 carbon atoms,
R² is hydrogen or an organic radical having 1 to 30 carbon atoms,
and the compound of the formula (I), after hydrolysis or enzymatic cleavage, releases an alcohol and a carboxylic acid in addition to the aldehyde or ketone; and
b) extracts from natural raw materials, hydrocarbons, aliphatic alcohols, aliphatic ketones, aliphatic nitriles, aliphatic carboxylic acids, acyclic terpene alcohols, acyclic terpene aldehydes, cyclic terpene aldehydes, cyclic alcohols, cycloaliphatic alcohols, cyclic or cycloaliphatic ethers, cyclic ketones, cycloaliphatic aldehydes or ketones, esters of cyclic alcohols or carboxylic acids, aromatic hydrocarbons, araliphatic alcohols, esters of araliphatic alcohols with aliphatic carboxylic acids, aromatic or araliphatic aldehydes, ketones or carboxylic acids, nitrogen-containing, aromatic compounds, phenols, phenyl ethers or phenyl esters, heterocyclic compounds or lactones.

6. Fragrances or perfume oils according to claim 5, wherein said fragrances or perfume oils are used undiluted in liquid form or diluted with a solvent.

7. Fragrances or perfume oils according to claim 5, wherein said fragrances or perfume oils are adsorbed to a carrier.

8. Fragrances or perfume oils according to claim 5, wherein said fragrances or perfume oils are microencapsulated or spray-dried or in the form of inclusion complexes or extrusion products.

9. Cosmetic care products comprising fragrances or perfume oils comprising
a) depot preparations comprising at least one compound of the formula (I) 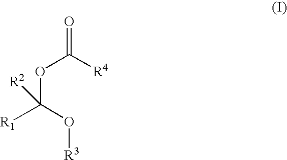
in which
R¹, R³ and R⁴, independently of one another, are an organic radical having 1 to 30 carbon atoms,
R² is hydrogen or an organic radical having 1 to 30 carbon atoms,
and the compound of the formula (I), after hydrolysis or enzymatic cleavage, releases an alcohol and a carboxylic acid in addition to the aldehyde or ketone; and
b) extracts from natural raw materials, hydrocarbons, aliphatic alcohols, aliphatic ketones, aliphatic nitriles, aliphatic carboxylic acids, acyclic terpene alcohols, acyclic terpene aldehydes, cyclic terpene aldehydes, cyclic alcohols, cycloaliphatic alcohols, cyclic or cycloaliphatic ethers, cyclic ketones, cycloaliphatic aldehydes or ketones, esters of cyclic alcohols or carboxylic acids, aromatic hydrocarbons, araliphatic alcohols, esters of araliphatic alcohols with aliphatic carboxylic acids, aromatic or araliphatic aldehydes, ketones or carboxylic acids, nitrogen-containing, aromatic compounds, phenols, phenyl ethers or phenyl esters, heterocyclic compounds or lactones.

10. Household products comprising fragrances or perfume oils comprising
a) depot preparations comprising at least one compound of the formula (I) 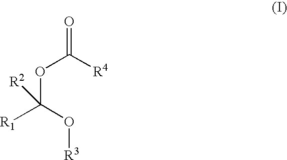
in which
R¹, R³ and R⁴, independently of one another, are an organic radical having 1 to 30 carbon atoms,
R² is hydrogen or an organic radical having 1 to 30 carbon atoms,
and the compound of the formula (I), after hydrolysis or enzymatic cleavage, releases an alcohol and a carboxylic acid in addition to the aldehyde or ketone; and
b) extracts from natural raw materials, hydrocarbons, aliphatic alcohols, aliphatic ketones, aliphatic nitriles, aliphatic carboxylic acids, acyclic terpene alcohols, acyclic terpene aldehydes, cyclic terpene aldehydes, cyclic alcohols, cycloaliphatic alcohols, cyclic or cycloaliphatic ethers, cyclic ketones, cycloaliphatic aldehydes or ketones, esters of cyclic alcohols or carboxylic acids, aromatic hydrocarbons, araliphatic alcohols, esters of araliphatic alcohols with aliphatic carboxylic acids, aromatic or araliphatic aldehydes, ketones or carboxylic acids, nitrogen-containing, aromatic compounds, phenols, phenyl ethers or phenyl esters, heterocyclic compounds or lactones.

11. Packaging products comprising fragrances or perfume oils comprising
a) depot preparations comprising at least one compound of the formula (I) 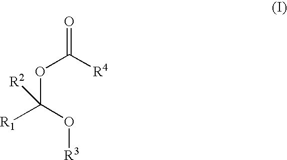
in which
R¹, R³ and R⁴, independently of one another, are an organic radical having 1 to 30 carbon atoms,
R² is hydrogen or an organic radical having 1 to 30 carbon atoms,
and the compound of the formula (I), after hydrolysis or enzymatic cleavage, releases an alcohol and a carboxylic acid in addition to the aldehyde or ketone; and
b) extracts from natural raw materials, hydrocarbons, aliphatic alcohols, aliphatic ketones, aliphatic nitriles, aliphatic carboxylic acids, acyclic terpene alcohols, acyclic terpene aldehydes, cyclic terpene aldehydes, cyclic alcohols, cycloaliphatic alcohols, cyclic or cycloaliphatic ethers, cyclic ketones, cycloaliphatic aldehydes or ketones, esters of cyclic alcohols or carboxylic acids, aromatic hydrocarbons, araliphatic alcohols, esters of araliphatic alcohols with aliphatic carboxylic acids, aromatic or araliphatic aldehydes, ketones or carboxylic acids, nitrogen-containing, aromatic compounds, phenols, phenyl ethers or phenyl esters, heterocyclic compounds or lactones.

12. Food products comprising fragrances or perfume oils comprising
a) depot preparations comprising at least one compound of the formula (I) 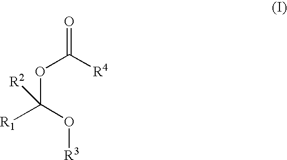
in which
R¹, R³ and R⁴, independently of one another, are an organic radical having 1 to 30 carbon atoms,
R² is hydrogen or an organic radical having 1 to 30 carbon atoms,
and the compound of the formula (I), after hydrolysis or enzymatic cleavage, releases an alcohol and a carboxylic acid in addition to the aldehyde or ketone; and
b) extracts from natural raw materials, hydrocarbons, aliphatic alcohols, aliphatic ketones, aliphatic nitriles, aliphatic carboxylic acids, acyclic terpene alcohols, acyclic terpene aldehydes, cyclic terpene aldehydes, cyclic alcohols, cycloaliphatic alcohols, cyclic or cycloaliphatic ethers, cyclic ketones, cycloaliphatic aldehydes or ketones, esters of cyclic alcohols or carboxylic acids, aromatic hydrocarbons, araliphatic alcohols, esters of araliphatic alcohols with aliphatic carboxylic acids, aromatic or araliphatic aldehydes, ketones or carboxylic acids, nitrogen-containing, aromatic compounds, phenols, phenyl ethers or phenyl esters, heterocyclic compounds or lactones.

13. Process for the preparation of depot preparations comprising at least one compound of the formula (I) 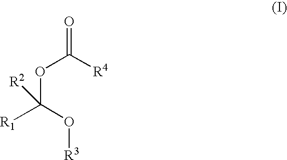
in which
R¹, R³ and R⁴, independently of one another, are an organic radical having 1 to 30 carbon atoms,
R² is hydrogen or an organic radical having 1 to 30 carbon atoms,
and the compound of the formula (I), after hydrolysis or enzymatic cleavage, releases an alcohol and a carboxylic acid in addition to the aldehyde or ketone;
comprising the steps of
i) reacting an aldehyde or a ketone with 2 to 4 equivalents of alcohol in the presence of catalytic amounts of acid to give the corresponding acetal or ketal then, under acidic catalysis,
ii) cleaving off one equivalent of alcohol from the acetal or ketal, thus giving the corresponding enol ether and subsequent acid-catalysed reaction of the enol ether with a carboxylic acid produces the compounds of formula (I).

14. Process for the preparation of depot preparations comprising at least one compound of the formula (I) 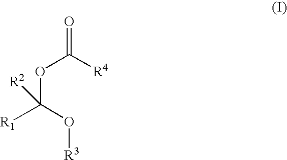
in which
R¹, R³ and R⁴, independently of one another, are an organic radical having 1 to 30 carbon atoms,
R² is hydrogen or an organic radical having 1 to 30 carbon atoms,
and the compound of the formula (I), after hydrolysis or enzymatic cleavage, releases an alcohol and a carboxylic acid in addition to the aldehyde or ketone;
comprising the steps of
producing the compounds of formula (I) by reacting a carboxylic ester or reaction of a carboxylic ester with an organometallic reagent and subsequent scavenging of the hemiacetal which forms with a carboxylic anhydride.
